(19) United States
(12) Patent Application Publication
(10) Pub. No.: US 2018/0147552 A1
(43) Pub. Date: May 31, 2018

(54) ZERO GRAVITY PROCESS DEVICE

(71) Applicant: Sarfaraz K. Niazi, Deerfield, IL (US)

(72) Inventor: Sarfaraz K. Niazi, Deerfield, IL (US)

(21) Appl. No.: 15/362,481

(22) Filed: Nov. 28, 2016

(51) Int. Cl. B01J 19/28; B01J 19/00; C12M 1/00

(52) U.S. Cl. CPC B01J 19/285; B01J 19/006; B01J 2219/00765; B01J 2219/00189; C12M 27/20

(57) ABSTRACT

A physical, chemical and biological process device operating under zero gravity and shaken by low energy mechanical and magnetic means is disclosed.

### BACKGROUND OF THE INVENTION

Physical, chemical and biological processes have in common the unit step of mixing the contents processed. The two common methods of mixing include a stirring device disposed in a stationary container or shaking, tumbling, rocking, vibrating or rotating a container holding the process contents. Using a stirring device is more popular in commercial process devices because it does not limit the size of container as it stays stationary. However, stirring devices are less desirable where the contents of the container need protecting from the environment and vice versa. Examples of processes that benefit from container mixing include biological processing and other situations where the container is kept sterile during processing, or where the use of stirred mixing damages the product because of high stress of stirred systems such as high molecular weight proteins in a bioprocess device.

Commercial processing by shaking a container remain limited because of the limitations of the physical, mechanical and financial resource required to shake the containers weighing into tons. There is an unmet need to create a technology to allow the use of small, low energy-consuming, small footprint devices to enable shaking a container to produce a variety of mixing profiles that reduce the cost of a process operations.

The instant invention provides a device that requires minimal force to operate by creating a zero-gravity effect on the container through hanging the container in the air or levitating it, and shaking the container by low-energy mechanical or electromagnetic devices to allow a plurality of mixing modes for optimal mixing.

### BRIEF SUMMARY OF THE INVENTION

The instant invention discloses a physical, chemical or biological process device comprising a container held in a zero-gravity state by magnetic levitation or by suspending it by hanging it in the air. Once the weight of the container is neutralized, it can be shaken by any available means including mechanical or magnetic means offering cost-effective solutions for a variety of industrial processing needs. The cost-effective outcome of the invention comes from the use of lowest power, smaller footprint devices that can easily fit in small places, more particularly in sterile facilities while offering a variety of mixing options and easy maintenance of equipment. The core element of the invention is the zero-gravity condition of the process device container; combining magnetic and mechanical means further allows a highly flexible configuration of the process device to meet specific needs of a broad category of processes.

The weight of an object is given by the mass multiplied by force (gravity). By levitating a container magnetically or by suspending a container in air removes the force and makes the object weightless. Shaking such container requires energy only to overcome the friction elements in shaking, which remains minimal. The cost of creating zero gravity is very low when using hanging means that have long repeat use life and when using magnetic levitation operated by small electrical power. Without first producing a zero gravity, the task of shaking the container will require high powered mechanical devices that are expensive and have a large footprint at the commercial scale processing level. Also, by creating a zero gravity on the container, it is possible to impart a variety of motion including horizontal and vertical simultaneously, to improve mixing not possible otherwise.

There is no prior art for a commercial process device operated in the state of levitation of suspension in the air.

There is no prior art of a process device that allows horizontal, vertical, orbital, vibratory mixing motions simultaneously to reduce the mixing times and to achieve optimal mixing. The de-mixing or segregation often seen in mixing operations is avoided by using multiple modes of mixing that reduce the mixing time and thus the segregation or de-mixing.

When suspending a container in the air, the vertical motion comes from a spring element connected between an anchor and the connecting means; when using magnetic levitation, this comes from cycling the magnetic field to levitate at different levels.

The process device disclosed is suitable for a variety of commercial operations including producing biological drugs.

To improve mixing, the container is provided with baffles in an arrangement that maximizes turbulence in the container.

An electronic controller is used to adjust the speed and frequency of shaking, as well as in producing magnetic fields necessary to levitate and shake the container. When the process device requires a flexible container, or where a plurality of container is used simultaneously, a platform is provided to hold the container and all elements of connectivity are added to the platform. The process device container may accommodate single-use bags or liners and may have a cylindrical or rectangular or square shape constituting a tote, a drum, or a box.

The process device can be a bioprocess device capable of holding a culture medium and a cell culture for producing a biological product, a chemical reaction vessel, a mixing vessel to dissolve solids into liquids, a fermenter for making beer and wines, or a variety of hundreds of commercial manufacturing operations.

Examples of physical processes include grinding of objects held in a container with abrasive elements, for polishing hard objects.

Some products such as alcohol products are stored on a long-term basis in containers that are tumbled frequently; the limitation of equipment available to tumble keeps the size of containers smaller, such as a barrel. However, with instant invention, these products can be aged in large containers reducing the cost of aging, and perhaps quality of aging.

Since the weight of the container is neutralized, any limits on the size and mass of the process device are removed. Also, one set of equipment can be used to operate process devices of different sizes, and even operate a plurality of process devices simultaneously on a platform, wherein a rack holding several process devices is placed on the platform.

The mechanical device to shake the process device container can be a motor driving a round chuck, which is connected to the underside or the side of the container through a set of gears to impart horizontal orbital motion to the container. The diameter of the chuck may be between 0.5 to 2 inches. Alternatively, the shaking device may comprise one or more magnets affixed to the side of the container and an equal number of stationary magnets facing the magnets affixed to the container. A sensor is provided to adjust magnetic forces to keep the shaking distance within a prescribed range.

The levitation inducing device may comprise of one or more magnets affixed to the underside of the container, and at least one stationary disposed below the underside of the container. When a plurality of magnets is affixed to the side of the process device container, and an equal number of fixed magnets are disposed facing these magnets on the container, it becomes possible to manipulate a variety of horizontal and orbital shaking of the container. When the shaking device employed is comprised of magnets, the anchor, the connectors, the container and the container are consisting of non-ferrous material.

Additionally, a set of vibrating elements can be attached to the process device to produce a mixing force in a variety of directions within the container.

The types of motions that can be imparted to the process device include horizontal shaking, vertical shaking, orbital motion, vertical vibrating, horizontal vibrating, rocking or a combination thereof.

### BRIEF DESCRIPTION OF THE SEVERAL VIEWS OF THE DRAWING

FIG. 1 shows a perspective view of a container disposed on a platform and connected to an anchor point using connection means.

FIG. 2 shows a perspective view of a container disposed on a platform and connected to an anchor point through a spring to allow a vertical motion.

FIG. 3 is a perspective view of a motorized means connected to the platform to make it shake vertically.

FIG. 4 is a perspective view of a motorized means connected to the platform to make it shake in an orbital motion.

FIG. 5 is a perspective view of a motorized means attached to the platform to make it shake horizontally.

FIG. 6 is a perspective view of a platform capable of levitation and shaking by magnetic means.

FIG. 7 is a perspective view of a rack to hold a plurality of containers.

### DETAILED DESCRIPTION OF THE INVENTION

Physical, chemical and biological processes at a commercial level inevitably requires mixing of the contents processed generally using an external stirring element introduced into a container holding the contents processed. An alternate method where the container is shaken is less desirable at a commercial level because of the tremendous weight of the container that requires expensive equipment, which is difficult to accommodate in particular manufacturing environments such as sterile rooms. To obviate the limitations of the use of external stirrers that expose the content to harm, the container mixing is preferred, and the instant invention offers a solution to all impediments in using container mixing in various types of processes.

Mixing technology is part of many commercial manufacturing operations in every industry. There are two options available to mix liquid and solid ingredients; one involved using an impeller to stir the components, and the another option is to rock, shake, vibrate, or rotate a container holding the components to be mixed; includes baffles in the wall of the container is used to improve the mixing efficiency. While both methods of mixing can provide a desirable mixing, there are reasons why one method may be preferred over the other method. The method using a stirring element is hard to employ in situations where a closed container is desirable or required. For example, continuous mixing is required in the operation of bioprocess devices, and historically the deep tank bioprocess devices are disposed with high-speed stirring elements. Recently, single-use promoted to reduce contamination risk. However, the technology has merely provided single-use liners to deep tank bioprocess devices. A desirable iteration of single-use technology will involve a single-use closed container, preferably a flexible container, that is devoid of any stirring element, allowing the bioprocess device to stay closed all times.

To shake the content of the container, it is rocked, horizontally shaken or subject to orbital shaking. The advantages of this design in bioprocess devices have great promise to allow manufacturing of affordable biological drugs, but it is severely constrained by the size of the container that can be used. Shaking a container weighing into tons requires large motors and other hardware that is difficult to accommodate in a clean room environment. Thus, these bioprocess devices have not been used for commercial manufacturing. There is an unmet need to provide means of shaking heavy containers to allow mixing of contents without employing large mechanical means.

The inertial mass measures an object's resistance to being accelerated (in this case shake) by force (represented by the relationship F=ma, where m is the mass, and a is the gravitational force). A higher force is required to overcome the inertial mass of the container, and as a result, a high force is provided using large motors. The instant invention provides means to reduce the inertial mass of the container by hanging the container in the air to an anchor through a plurality of connectors wherein the stress transfers to the connectors neutralizing the force of gravity. The same results can be achieved by magnetically levitating the container.

Once the weight of the container is neutralized, it becomes possible to shake the container in any direction with minimal force, which can be mechanical such as using a motor and set of gears or magnetic to achieve a variety of shaking shakings. Magnetic levitation and magnetic shaking involve disposing a magnet to the container and a magnet disposed at a stationary surface facing the magnet disposed on the container. A change in the magnetic force or polarity by electrical means produces levitation or shaking of the container.

A shaking of the container is also possible by a vibrator attached to the container without bearing the mass of the container. The vibrator can be a mechanical vibrator or an ultrasonic or sonic vibrator.

The net result of employing the described features of the invention is reduction capital requirements, lowest operating costs and high quality of products produced in a variety of physical, chemical and biological industries.

The absence of large mechanical devices allows the container to be disposed closer to the ground, reducing the center of gravity and thus larger volume containers can be used in rooms with limited ceiling height, keeping the footprint of the device to a minimum and the cost of the operations at the lowest level. For example, a six-cubic-foot tote allows about 6000 liters of liquid or solids to be mixed in a standard 10-foot ceiling height room while occupying only a 6×6 ft. footprint. This operation is not possible if the traditional technology is shaking containers that are fully subject to gravitational force.

The type of mixing that can benefit from the instant invention includes chemical process devices, abrasive polishing, grinding, dissolving solids in a solution, product aging, pharmaceutical batch processing of powders and liquids, bioprocessing and a variety of other situations where a mixing element inside the container is not desirable or where a closed operation is desired to protect the product, the environment or the operators.

One example of processing that can substantially benefit from the instant invention is in bioprocessing to grow living entities such as bacteria, mammalian cells, plant cells, viruses, etc., to manufacture chemicals and drugs of interest to humans and animals. Bioprocess devices are containers to hold a nutrient media and a biological culture that is agitated vigorously, particularly when a bacterium is grown, to dilute the metabolic chemicals and to impart energy to force dissolved gasses like carbon dioxide to leave the liquid phase.

The agitation of the content of a bioprocess device is achieved mostly by introducing a stirring stirrer that operates at a very high speed to generate a turbulent motion in the container that is not possible if a container is itself shaken. Whereas for decades, this design of bioprocess devices has worked well leading to an industry that spans multi-billion dollar markets, over the past couple of decades, a new class of drugs have been developed that come from recombinant cells and bacteria. These are large molecular weight proteins that are highly subject to structural modification if the stress to cells and the proteins is high in the container.

This realization requires the development of bioprocess devices that are not stirred by an external stirrer and further to avoid contamination. However, without an external stirrer, the container such as a bioprocess devices must be agitated in their entire body to achieve a similar level of mixing as obtained when using stirrers in small-scale bioprocess devices such as a shake flask, but when the volume of liquid is into thousands of liters, this requires construction of highly complex and large equipment to provide the shaking. The instant invention resolves these impediments.

The mixing of contents of the container improves by further introducing baffles in the container.

The container can be hard-walled or a soft-walled single-use sterile container.

The container can be disposed on a solid platform to allow the use of multiple size containers on the same platform.

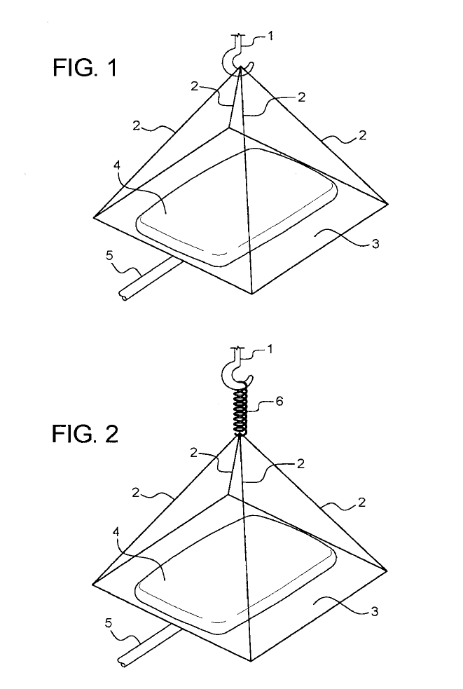

The preferred suspended mixing assemblies are illustrated in FIGS. 1-7.

FIG. 1 shows an anchor (1) disposed above the level of a platform (3) holding a container (4), a set of connectors (2) connecting the platform to the anchor and a mechanical shaft (5) connecting the platform a motor capable of imparting motion to the platform. In the absence of the platform, the mechanical shaft connects connect directly to the container. The container is shown is a flat bag but there is no restriction on the shape of the container.

FIG. 2 shows a spring (6) disposed between the anchor and the connecting means to allow a vertical motion to the platform. The spring's force is the force exerted by a compressed or stretched spring upon any object that is attached to it. An object that compresses or stretches a spring is always acted upon by a force that restores the object to its rest or equilibrium position. For most springs (specifically, for those that are said to obey “Hooke's Law”), the magnitude of the force is directly proportional to the amount of stretch or compression of the spring. By attaching a platform to at least one spring, the weight of the container becomes easily pushed up or down. The vertical shaking of the container ranges from 1-5 inches, most desirably, 2 inches, for most optimal mixing, since the purpose is to provide a repeated cycle; longer swings has a smaller frequency.

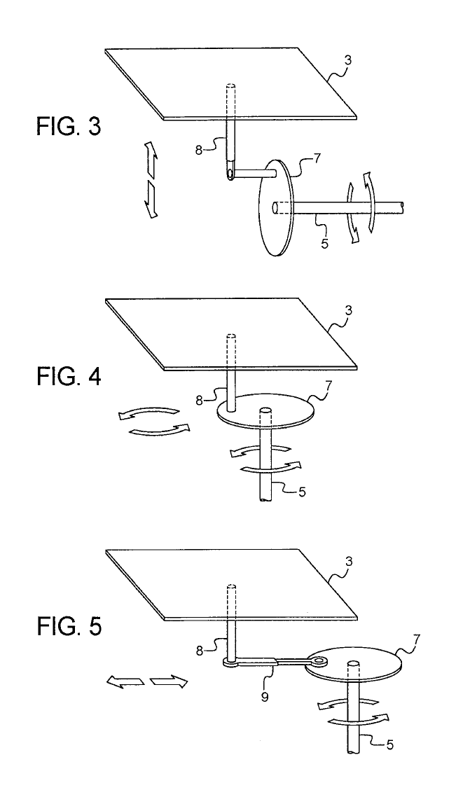

FIG. 3 shows a mechanical arrangement connected to platform through a vertical connector (8), which connects to a chuck (7) further connected to a motor (not shown) to a shaft (5). FIGS. 1 and 2 show the shaft (5) since the mechanical connection shown in FIG. Three hide under the platform in FIGS. 1 and 2. The mechanical means shown here are mere as an example as many other arrangements to impart motion to the platform are possible, and the instant invention does not limit the scope of these means.

FIG. 4. shows a perspective view the mechanical means shown in FIG. 3 with a change in the orientation of the chuck (7) to impart an orbital motion to the platform; discussion of limitations as described applies to this arrangement as well.

FIG. 5. shows a perspective view the mechanical means shown in FIG. 4 with an additional connector (9) that provides a horizontal motion to the platform; discussion of limitations as described applies to this arrangement as well.

The shaking assemblies as shown in FIGS. 3-5 demonstrates how a large mass moves easily. The size of the chuck may range from 0.5 to 10 inches, but preferably, it is between 1 and 2 inches, and more preferably, 2 inches in diameter. By placing the motor sideways or on the top of the platform, it is possible to lower the level of the platform thereby lowering the center of gravity that provides additional stability to the equipment. There may be additional gears placed between the motor and the chuck to increase the torque of the chuck. It is noteworthy that the chuck does not bear any weight of the container, and the torque of the chuck provides only the kinetic energy and a small amount of potential energy where the container rises in a swing, albeit at a tiny angle when using a 2-inch chuck.

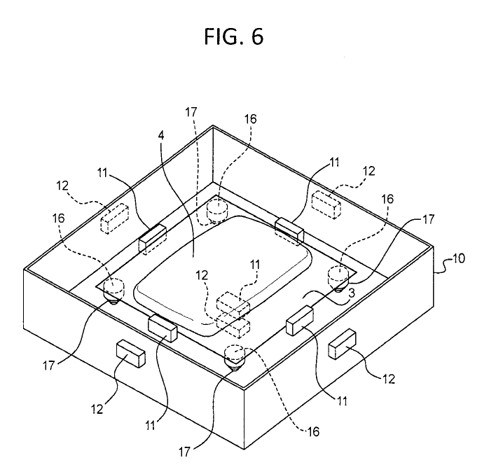

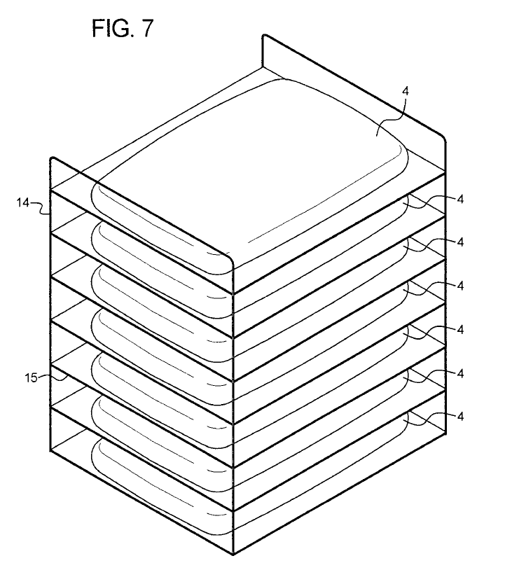

FIG. 6 shows a surrounding stationary surface (10) surrounding the platform, the magnets connected to platform (11), the magnets connected to stationary surfaces (12) intended to induce horizontal and horizontal shaking, a vibrator connector (16) disposed on the underside of the platform and a vibrator (17) disposed on the underside surface of the platform; alternately or in addition, the vibrator can be connected to the side surface of the platform as well.

To employ a magnetic shaking of the platform, there is a need to introduce a stationary surface surrounding the platform that will hold the magnets, opposing the magnets connected to the container surfaces. FIG. 6 depicts an arrangement of magnets on the platform or the container and magnets affixed to stationary surfaces. The surfaces of the container and/or the stationary surface can be equipped with an electronic sensor to detect the distance between the surfaces of the container and the stationary surface to allow maintaining a pre-determined distance continuously or periodically, as required, throughout the mixing process.

The material of construction of the platform, the container, and the connectors is preferably non-ferrous when using electromagnets.

An electromagnet is a type of magnet wherein an electric current produces the magnetic field. The magnetic field disappears when the current is turned off Electromagnets usually consist of many closely spaced turns of wire that create the magnetic field. The wire turns are often wound around a magnetic core made from a ferromagnetic or ferromagnetic material such as iron. The magnetic core concentrates the magnetic flux and makes a more powerful magnet. The main advantage of an electromagnet over a magnet is that the magnetic field can be quickly changed by controlling the amount of electric current in the winding. Electromagnets are widely used as components of other electrical devices, such as motors, generators, relays, loudspeakers, hard disks, MRI machines, scientific instruments, and magnetic separation equipment. In the instant invention, electromagnets are used to impart a variety of motions of the container. To provide a horizontal linear motion, at least one electromagnet is affixed to the edge of the container and at least one stationary electromagnet affixed on the container. Both magnets are turned on to produce an opposite polarity, causing the container to swing towards the stationary magnet. At least one of the electromagnet assemblies may, optionally, can measure the distance between the two magnets via a sensor. When the container/container reaches a predetermined distance from the stationary electromagnet, the polarity of either the electromagnet affixed to the container/container or the stationary electromagnet switches creating a repelling force that pushes the container back. Given that the container is hanging like a pendulum, it will move back beyond its starting position and return in a swing oscillation. However, to keep the swing constant, the magnets switch the polarity to attraction mode forcing the container to reach a pre-determined distance to the stationary magnet. This arrangement assures that the arc of the swing is always maintained constant. Alternately, a similar set of electromagnets can be affixed to the opposite side of the container to perform the similar attraction and repelling function to make sure that the arc of the swing is identical on both side. To create an orbital motion, at least three sets of electromagnets are required, disposed at an equal distance from each other if the shape of the container is round. In the case, the container is rectangular or square in shape having four edges, and then, at least, four sets of magnets are used. The attraction and repelling cycle are programmed to move from one set of electromagnets to another set of electromagnets in a clockwise or anticlockwise direction to create an orbital oscillation of the container. To create a vertical motion, at least, one electromagnet attached to the underside of the container and, at least, one stationary electromagnet affixed facing the electromagnet affixed to the container. The motion is started by creating an opposite polarity to push the container upward, which is expedited by the strain on the spring and switched off when the container reaches a pre-determined distance to the stationary electromagnet. This causes the container to swing upward based on its mass and the tension in the spring. The container then returns downward and is attracted by the magnet to the same distance. The electromagnets can be programmed to attract or repel between the container and the stationary surface as well as to measure the distance between the electromagnets by an electronic controller.

A vertical motion of the container can be achieved by changing the level of levitation to a higher and a lower level alternately.

FIG. 7 shows a perspective view of a rack (14) with shelves (15) holding a plurality of containers (4). In creating a commercially feasible manufacturing operation, processing of larger volumes of liquids or solids at one time is desirable. When using flexible containers, there can be a limitation of size for a single container, which may have the limited strength to hold liquids and solids. This obstacle can be overcome by stacking a plurality of containers in a rack. Given that the weight of the container is not an impediment, the number of containers that are stacked will be determined by the height of the ceiling in a room. For most standard 10-ft ceiling clearance manufacturing rooms, one can easily stack 5-6 containers, allowing at least 12 inches between them.

Since the main impediment to shaking a heavy objects is simplified and made easier in the instant invention, there is no restriction on the volume of mixing that can be achieved using the instant invention. Larger volumes of mixing substances can be divided into multiple containers if there are limitations on the size or the footprint of the container.

Containers may be of any size, e.g., 5 liters, 10 liters, 100 liters, 250 liters, 500 liters, 1000, liters, etc., and or shape, e.g., rectangular, square, ovoid, circular.

In one embodiment, the instant invention comprises a container held in a state of zero gravity by suspending the container using a plurality of connectors between the container and an anchor located above the container, which is shaken by mechanical or magnetic means to induce horizontal or orbital shaking.

In another embodiment, the instant invention comprises a container held in a state of zero gravity by suspending the container using a plurality of connectors and at least one spring between the container and an anchor located above the container, which is shaken by mechanical or magnetic means to induce an oscillating vertical shaking.

In another embodiment, the instant invention comprises a container held in a state of suspension by magnetic levitation and the container is shaken by mechanical or magnetic means.

In another embodiment, the instant invention has multiple containers disposed on a rack.

In another embodiment, the instant invention describes a container that is a bioprocess device.

In another embodiment, the instant invention a container that is a single-use bioprocess device.

In another embodiment, the instant invention is a mixing device, a bioprocess device, a chemical synthesizing device, a chemical reaction device, a grinding device, a polishing device, an organ growth device, or a product aging device.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein is merely intended to serve as a shorthand method of referring individually to each separate value falling within the range unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate better the invention and does not pose a limitation on the scope of the invention unless otherwise claimed. No language in the specification should be construed as indicating any non-claimed element as essential to the practice of the invention.

What is claimed is:

1. A physical, chemical or biological process device operating under conditions of zero gravity, comprising;
a container with an underside, at least one side, and an inner volume to hold a liquid, a solid, a biological entity, or a mixture thereof;
a means for levitating or suspending the container in the air;
a magnetic or a mechanical means for repetitively shaking the container; and
a means for controlling the repetitive shaking of the container.

2. The device of claim 1, wherein the process device is a mixing device, a bioprocess device, a chemical synthesis device, a chemical reaction device, a grinding device, a polishing device, an organ growth device, or a product aging device.

3. The device of claim 1, wherein shaking comprises horizontal linear shaking, horizontal orbital shaking, vertical shaking, vibrating, rocking the container, or a combination thereof

4. The device of claim 1, wherein the means for suspending the container comprise a plurality of solid connectors including a string, rope, a chain, wire, spring, or a combination thereof, connecting the container to at least one anchor point fixed above the container.

5. The device of claim 1, wherein the means for levitating the container comprise at least one magnet disposed on the underside of the container and one magnet disposed on a stationary surface facing the underside of the container and an electrical means for creating a magnetic field of sufficient strength between the two magnets to cause the container to levitate.

6. The device of claim 1, wherein the container and any attachments to the container are of non-ferrous properties when any magnetic means are employed in the operation of the device.

7. The device of claim 5, wherein the container levitates to a height of 0.1 inches to 2 inches from the stationary surface.

8. The device of claim 5, wherein the container levitates alternately to at least two different heights to induce a vertical shaking to the container.

9. The device of claim 1, wherein the magnetic means for shaking the container comprise at least one magnet disposed on the side of the container and at least one magnet disposed on a stationary surface facing the side of the container and an electrical means for creating a magnetic force between the magnets sufficient to create a linear horizontal or orbital horizontal shaking of the container within a pre-determined range.

10. The device of claim 1, wherein the means for shaking the container comprise a mechanical assembly comprising a motor, a round chuck, and a shaft connected to the underside of the container to induce linear horizontal, orbital horizontal and vertical shaking of the container and an electronic means to control the amplitude and frequency of shaking within a pre-determined range.

12. The device of claim 1, wherein the diameter of the chuck ranges between 0.5 to 2 inches.

13. The device of claim 1, wherein the magnetic means comprises magnets that are permanent magnets or electromagnets, or a combination thereof.

14. The device of claim 1, wherein the mechanical means of shaking the container comprise a mechanical, sonic or ultrasonic vibrating element in contact with the or the side or the container and an electronic means for controlling frequency and intensity of vibration to induce continuous vibration to the container.

15. The device of claim 1, wherein the container further comprises at least one baffle surface disposed inside the container.

16. The device of claim 1, wherein the container further comprises a solid platform to hold the container, and all means for levitating, suspending and shaking are in communication with the platform.

17. The device of claim 16, wherein a rack is disposed on the platform holding a plurality of containers.
